(19) United States
(12) Patent Application Publication
(10) Pub. No.: US 2013/0274294 A1
(43) Pub. Date: Oct. 17, 2013

(54) 4-(Hetero)Aryl-Ethynyl-Octahydro-Indole-1-Esters

(71) Applicants: David Carcache, Binningen (CH); Manuel Koller, Schliern (CH); Silvio Ofner, Muenchenstein (CH); David Orain, Hesingue (FR); Johanne Renaud, Quebec (CA)

(72) Inventors: David Carcache, Binningen (CH); Manuel Koller, Schliern (CH); Silvio Ofner, Muenchenstein (CH); David Orain, Hesingue (FR); Johanne Renaud, Quebec (CA)

(21) Appl. No.: 13/995,282

(22) PCT Filed: Dec. 19, 2011
(86) PCT No.: PCT/EP2011/073304

§ 371 Date: Jun. 18, 2013

Related U.S. Application Data
(60) Provisional application No. 61/425,127, filed on Dec. 20, 2010.

(51) Int. Cl. C07D 209/08; C07D 417/08; A61K 31/427; A61K 31/403; A61K 31/4439; C07D 401/08; A61K 45/06

(52) U.S. Cl. CPC C07D 209/08; C07D 401/08; C07D 417/08; A61K 45/06; A61K 31/403; A61K 31/4439; A61K 31/427
USPC 514/339; 548/452; 546/278.1; 548/181; 514/412; 514/365

(57) ABSTRACT

The invention relates to compound of the formula (I) or a salt thereof, wherein the substituents are as defined in the specification; to its preparation, to its use as medicament and to medicaments comprising it.

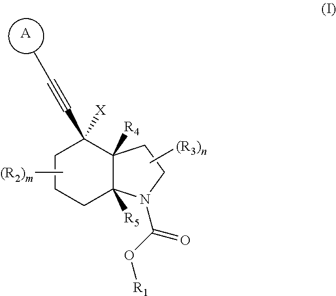

The invention relates to 4-(Hetero)aryl-ethynyloctahydro-indole-1-esters, to their preparation, to their use as medicaments and to medicaments comprising them.

Some mGluR5 antagonists are described e.g. in WO2003047581 and WO2010018154.

mGluR5 antagonists are considered to be useful in the treatment of a wide range of disorders, in particular fragile X syndrome (FXS), L-dopa induced dyskinesias in Parkinsons Disease (PD-LID) and Gastro-Esophageal Reflux Disease (GERD).

There is a need to provide new mGluR5 antagonists that are good drug candidates. In particular, preferred compounds should bind potently to mGluR5 whilst showing little affinity for other mGluRs. They should exhibit a low binding to plasma proteins. They should be well absorbed from the gastrointestinal tract, be sufficiently metabolically stable and possess favorable pharmacokinetic properties. They should be non-toxic and demonstrate few side-effects. Furthermore, the ideal drug candidate will be able to exist in a physical form that is stable, non-hygroscopic and easily formulated. Especially suitable drug candidates for pediatric uses (e.g. with infant FXS patients) have a good solubility in water; this facilitates manufacture of products being liquid drinking formulations and/or solid formulations for making liquid drinking products. A favorable pharmacokinetic profile for an oral-once-daily administered product shows a long-lasting blood exposure of the active principle after oral administration while avoiding an overly high maximum plasma concentration (cₘₐₓ).

The compounds of the invention are mGluR5 antagonists and are therefore potentially useful in the treatment of a wide range of disorders, particularly FXS, PD-LID and GERD.

In a first aspect, the invention relates to a compound of the formula I

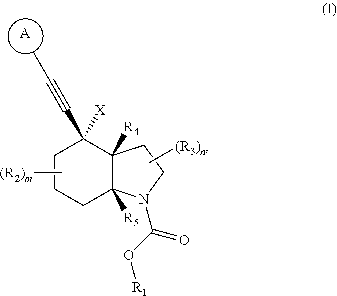

or a salt thereof, wherein
R₁ is C₁₋₄alkyl, C₃₋₆cycloalkyl or C₃₋₆cycloalkyl-C₁₋₄alkyl;
R₂ and R₃ independently are halogen, cyano, hydroxy, amino, C₁₋₄alkyl; C₁₋₄halogenalkyl; C₁₋₄hydroxyalkyl; C₁₋₄-aminoalkyl; C₁₋₄alkylamino-C₁₋₄alkyl; di-(C₁₋₄alkyl)amino-C₁₋₄alkyl; C₁₋₄alkoxy-C₁₋₄alkyl; C₂₋₄alkenyl; C₂₋₄halogenalkenyl; C₂₋₄alkinyl; C₂₋₄halogenalkinyl; C₁₋₄alkoxy; C₁₋₄halogenalkoxy; di-(C₁₋₄alkyl)amino or C₃₋₆cycloalkyl, wherein one carbon atom of the C₃₋₆cycloalkyl may be replaced by an oxygen atom and wherein the C₃₋₆cycloalkyl may be attached directly to the ring system or via a C₁₋₂alkylene or an oxygen;
m is 0, 1, 2, 3, 4, 5 or 6;
n is 0, 1, 2, 3 or 4;
R₄ and R₆ independently are hydrogen, halogen or methyl;
X is hydroxy or amino;
A is selected from

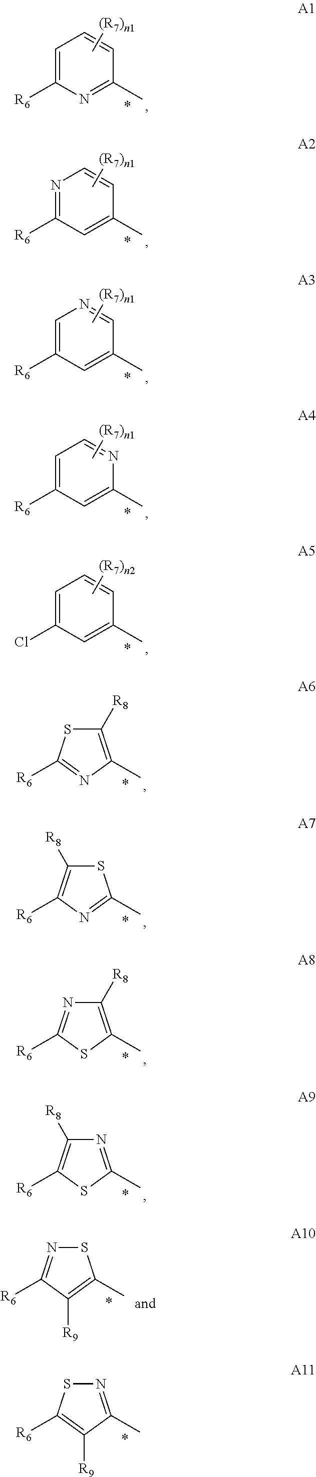

wherein the bond marked with the asterisk is attached to the ethynyl-moiety;
R₆ is hydrogen, halogen, methyl or halogenmethyl;
R₇ is halogen, hydroxy, amino, cyano, methyl or methoxy;
R₆ is hydrogen, halogen, hydroxy, amino, cyano, methyl or methoxy;
n1 is 0, 1, 2 or 3; and
n2 is 0, 1, 2, 3 or 4.

Unless specified otherwise, the term “compounds of the invention” refers to compounds of formula (I) and subformulae thereof; salts of the compounds; hydrates or solvates of the compounds and/or salts; as well as all stereoisomers (including diastereoisomers), tautomers and isotopically labeled compounds (including deuterium substitutions); as well as inherently formed moieties (e.g. polymorphs, solvates and/or hydrates).

Unless indicated otherwise, the expressions used in this invention have the following meaning:

“Alkyl” represents a straight-chain or branched-chain alkyl group and, for example, may be methyl, ethyl, n- or iso-propyl, n-, iso-, sec- or tert-butyl; C₁₋₄alkyl typically represents a straight-chain or branched-chain C₁₋₃alkyl, e.g. methyl, ethyl, n-propyl or iso-propyl.

Each alkyl part of “alkoxy”, “halogenalkyl” and so on shall have the same meaning as described in the above-mentioned definition of “alkyl”, especially regarding linearity and size.

“C₃₋₆cycloalkyl” represents a saturated alicyclic moiety having from three to six carbon atoms. This term refers to groups such as cyclopropyl, cyclobutyl, cyclopentyl and cyclohexyl.

Halogen is generally fluorine, chlorine, bromine or iodine; e.g. fluorine, chlorine or bromine. Halogenalkyl groups typically have a chain length of 1 to 4 carbon atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl.

Depending on further substituent definitions, the compounds of formula I may exist in optically active form or in form of mixtures of optical isomers, e.g. in form of diastereomeric mixtures. In particular, further asymmetrical carbon atom(s) may be present in the compounds of formula I and their salts. Unless otherwise provided herein, all such optical isomers and their mixtures are embraced by the invention. In this context it is noted that for all compounds of formula I, the asymmetrical carbon atoms in the 3a-, 4- and 7a-position of the octahydro-indole-moiety have the fixed configuration as shown in the image for compounds of formula I. For example, compounds of formula I, wherein R₄ and R₅ are both hydrogen, have a (3aR,4S,7aR)-configuration.

As used herein, the term “isomers” refers to different compounds that have the same molecular formula but differ in arrangement and configuration of the atoms.

Also as used herein, the term “an optical isomer” or “a stereoisomer” refers to any of the various stereo isomeric configurations which may exist for a given compound of the invention.

It is understood that a substituent may be attached at a chiral center of a carbon atom. The term “chiral” refers to molecules which have the property of non-superimposability on their mirror image partner, while the term “achiral” refers to molecules which are superimposable on their mirror image partner.

“Enantiomers” are a pair of stereoisomers that are non-superimposable mirror images of each other.

A 1:1 mixture of a pair of enantiomers is a “racemic” mixture. The term is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or depending on the direction (dextro- or levorotatory) which they rotate plane polarized light at the wavelength of the sodium D line.

The compounds described herein may contain—besides the three centers depicted for compounds of formula I—one or more further asymmetric centers and may thus give rise to diastereomers, and other stereoisomeric forms that may be defined, in terms of absolute stereochemistry, as (R)- or (S)-. Unless otherwise provided herein, the invention is meant to include all such possible isomers, including mixtures and optically pure forms.

Optically active (R)- and (S)-isomers may be prepared using chiral synthons or chiral reagents, or resolved using conventional techniques.

If the compound contains a double bond, substituent(s) may have an E or Z configuration.

Unless provided otherwise herein, any asymmetric atom (e.g. carbon or the like) of the compound(s) of the invention may be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration.

Substituents at atoms with unsaturated bonds may, if possible, be present in cis-(Z)- or trans-(E)-form.

Accordingly, as used herein and unless provided otherwise herein, a compound of the invention can be in the form of one of the possible isomers, rotamers, atropisomers, tautomers or mixtures thereof, for example, as substantially pure geometric (cis or trans) isomers, diastereomers, optical isomers (antipodes) or mixtures thereof.

Unless provided otherwise herein, any resulting mixtures of isomers can be separated on the basis of the physicochemical differences of the constituents, into the pure or substantially pure geometric or optical isomers, diastereomers, for example, by chromatography and/or fractional crystallization.

Unless provided otherwise herein, any resulting mixtures of isomers of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Mixtures of isomers can also be resolved by chiral chromatography, e.g., high pressure liquid chromatography (HPLC) using a chiral adsorbent.

Depending on substituent definition, compounds of formula I may occur in various tautomeric forms. All tautomeric forms of the compounds of formula I are embraced by the invention.

As used herein, the terms “salt” or “salts” refers to an acid addition or base addition salt of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. The compounds of the invention may be capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

When both a basic group and an acid group are present in the same molecule, the compounds of the invention may also form internal salts, e.g., zwitterionic molecules.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁸Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula I with the co-crystal former under crystallization conditions and isolating co-crystals thereby formed. Suitable co-crystal formers include those described in WO 2004/078163. Hence the invention further provides co-crystals comprising a compound of formula (I).

The invention also envisages the use of pro-drugs of the compounds of the invention that convert in vivo to the compounds of the invention. A pro-drug is an active or inactive compound that is modified chemically through in vivo physiological action, such as hydrolysis, metabolism and the like, into a compound of the invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001).

Furthermore, the compounds of the invention, including their salts, can also be obtained in the form of their hydrates, or include other solvents used for their crystallization. The compounds of the invention may inherently or by design form solvates with pharmaceutically acceptable solvents (including water); therefore, it is intended that the invention embrace both solvated and unsolvated forms. The term “solvate” refers to a molecular complex of a compound of the invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the invention, including salts, hydrates and solvates thereof, may inherently or by design form polymorphs.

Preferred substituents, preferred ranges of numerical values or preferred ranges of the radicals present in compounds of the formula I and the corresponding intermediate compounds are defined below. The definition of the substituents applies to the end-products as well as to the corresponding intermediates. The definitions of the substituents may be combined at will, e.g. preferred substituents R₁ and particularly preferred substituents R₂.

In one embodiment, the invention provides a compound of formula I or a salt thereof, wherein R₁ is C₁₋₄alkyl.

In one embodiment, the invention provides a compound of formula I or a salt thereof, wherein R₁ is methyl.

In one embodiment, the invention provides a compound of formula I or a salt thereof, wherein R₁ is ethyl.

In one embodiment, the invention provides a compound of formula I or a salt thereof, wherein m is 0, 1, 2 or 3; and n is 0, 1 or 2.

In one embodiment, the invention provides a compound of formula I or a salt thereof, wherein R₂ and R₃ independently are halogen, cyano, hydroxy, amino, C₁₋₄alkyl; C₁₋₄halogenalkyl or C₁₋₄alkoxy;

m is 0, 1, 2 or 3; and
n is 0, 1 or 2.

In one embodiment, the invention provides a compound of formula I or a salt thereof, wherein m and n are both 0.

In one embodiment, the invention provides a compound of formula I or a salt thereof, wherein R₄ and R₅ are both hydrogen.

In one embodiment, the invention provides a compound of formula I or a salt thereof, wherein X is hydroxy.

In one embodiment, the invention provides a compound of formula I or a salt thereof, wherein X is amino.

In one embodiment, the invention provides a compound of formula I or a salt thereof, wherein A is A1.

In one embodiment, the invention provides a compound of formula I or a salt thereof, wherein A is A2.

In one embodiment, the invention provides a compound of formula I or a salt thereof, wherein A is A2; wherein the bond marked with the asterisk is attached to the ethynyl-moiety; R₆ is hydrogen, halogen, methyl, fluoromethyl, difluoromethyl or trifluoromethyl; R₇ is fluoro; and n1 is 0, 1 or 2.

In one embodiment, the invention provides a compound of formula I or a salt thereof, wherein A is A2; wherein the bond marked with the asterisk is attached to the ethynyl-moiety; R₆ is chloro, methyl, fluoromethyl, difluoromethyl or trifluoromethyl; R₇ is fluoro; and n1 is 0, 1 or 2.

In one embodiment, the invention provides a compound of formula I or a salt thereof, wherein A is A2; wherein the bond marked with the asterisk is attached to the ethynyl-moiety; R₆ is chloro or methyl; R₇ is fluoro; and n1 is 0, 1 or 2.

In one embodiment, the invention provides a compound of formula I or a salt thereof, wherein A is A2; wherein the bond marked with the asterisk is attached to the ethynyl-moiety; R₆ is chloro or methyl; and n1 is 0.

In one embodiment, the invention provides a compound of formula I or a salt thereof, wherein A is A2; wherein the bond marked with the asterisk is attached to the ethynyl-moiety; R₆ is chloro; and n1 is 0.

In one embodiment, the invention provides a compound of formula I or a salt thereof, wherein A is A2; wherein the bond marked with the asterisk is attached to the ethynyl-moiety; R₆ is methyl; and n1 is 0.

In one embodiment, the invention provides a compound of formula I or a salt thereof, wherein A is A3.

In one embodiment, the invention provides a compound of formula I or a salt thereof, wherein A is A4.

In one embodiment, the invention provides a compound of formula I or a salt thereof, wherein A is A5.

In one embodiment, the invention provides a compound of formula I or a salt thereof, wherein A is A5; wherein the bond marked with the asterisk is attached to the ethynyl-moiety; R₇ is fluoro; and n1 is 0, 1 or 2.

In one embodiment, the invention provides a compound of formula I or a salt thereof, wherein A is A5; wherein the bond marked with the asterisk is attached to the ethynyl-moiety; and n1 is 0.

In one embodiment, the invention provides a compound of formula I or a salt thereof, wherein A is A6.

In one embodiment, the invention provides a compound of formula I or a salt thereof, wherein A is A7.

In one embodiment, the invention provides a compound of formula I or a salt thereof, wherein A is A8.

In one embodiment, the invention provides a compound of formula I or a salt thereof, wherein A is A9.

In one embodiment, the invention provides a compound of formula I or a salt thereof, wherein A is A10.

In one embodiment, the invention provides a compound of formula I or a salt thereof, wherein A is A11.

In one embodiment, the invention provides a compound of formula I or a salt thereof, wherein

R₁ is C₁₋₄alkyl;
m and n are both 0;
R₄ and R₅ are both hydrogen;
X is hydroxy;
A is A2; wherein the bond marked with the asterisk is attached to the ethynyl-moiety;
R₆ is chloro, methyl, fluoromethyl, difluoromethyl or trifluoromethyl;
R₇ is fluoro;
and n1 is 0, 1 or 2.

In one embodiment, the invention provides a compound of formula I or a salt thereof, wherein

R₁ is C₁₋₄alkyl;
m and n are both 0;
R₄ and R₅ are both hydrogen;
X is hydroxy;
A is A5; wherein the bond marked with the asterisk is attached to the ethynyl-moiety;
R₇ is fluoro; and n1 is 0, 1 or 2.

In a further aspect, the invention also provides a process for the production of compounds of the formula Ia. Compounds of the formula Ia are obtainable according to the following process as described in scheme 1:

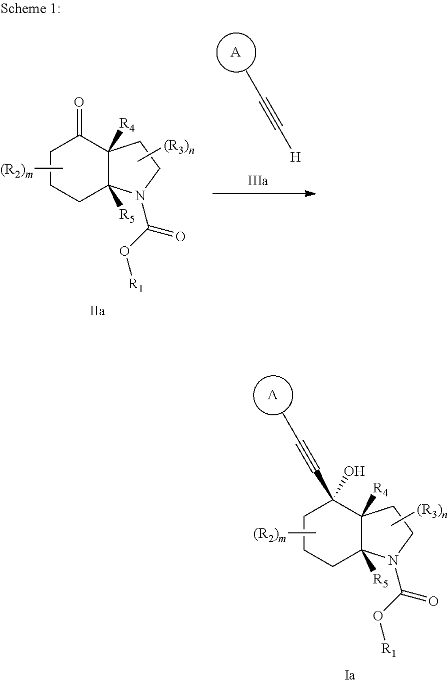

A compound of formula Ia, in which R₁, R₂, R₃, R₄, R₅, m, n and A are as defined under formula I, may be obtained by reacting compound of formula IIa, in which R₁, R₂, R₃, R₄, R₅, m and n are as defined under formula I, with a compound of formula IIIa, in which A is as defined under formula I, in the presence of a suitable base, e.g. butyl lithium or lithium diisopropylamide, in the presence of a suitable solvent, e.g. an suitable anhydrous solvent, such as anhydrous tetrahydrofurane or anhydrous diethylether.

In a further aspect, the invention also provides an alternative process for the production of compounds of the formula Ia. Compounds of the formula Ia are obtainable according to the following process as described in scheme 2:

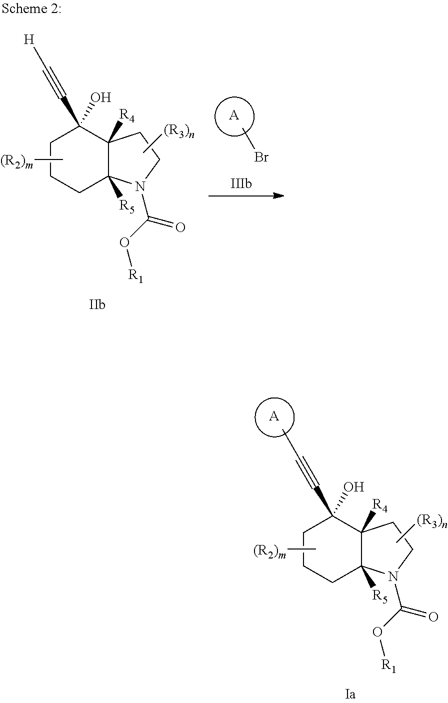

A compound of formula Ia, in which R₁, R₂, R₃, R₄, R₅, m, n and A are as defined under formula I, may be obtained by reacting compound of formula IIb, in which R₁, R₂, R₃, R₄, R₅, m and n are as defined under formula I, with a compound of formula IIIb, in which A is as defined under formula I, in the presence of a suitable base, e.g. triethyl amine, in the presence of a suitable solvent, e.g. anhydrous dimethylformamide, and a suitable catalyst, e.g. CuI and Pd(PPh₃)₄.

Further compounds of formula I or their precursors may be obtainable from compounds of formula Ia or their precursors (e.g. compounds of formulae IIa and/or IIb)—prepared as described according to scheme 1 or scheme 2—by reduction, oxidation and/or other functionalization of resulting compounds and/or by cleavage of any protecting group(s) optionally present, and of recovering the so obtainable compound of the formula I. Compounds of the formula I can also be prepared by further conventional processes, e.g. as described in the Examples, which processes are further aspects of the invention.

The reactions can be effected according to conventional methods, for example as described in the Examples.

The work-up of the reaction mixtures and the purification of the compounds thus obtainable may be carried out in accordance with known procedures.

Acid addition salts may be produced from the free bases in known manner, and vice-versa.

Starting materials, e.g. compounds of the formulae IIa, IIb, IIIa and IIIb are known (e.g. from WO2010018154) or may be prepared according to conventional procedures starting from known compounds, for example as described in the Examples.

In a further aspect, the invention provides a process for the production of a compound of the formula Ia

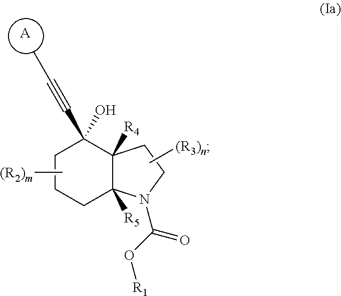

in which R₁, R₂, R₃, R₄, R₅, m, n and A are as defined under formula I,
or a salt thereof,
which comprises reacting a compound of formula IIa

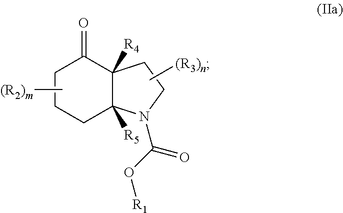

in which R₁, R₂, R₃, R₄, R₅, m and n are as defined under formula I,
or a salt thereof,
with a compound of formula IIIa

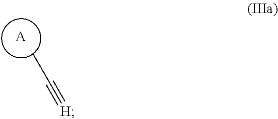

in which A is as defined under formula I,
or a salt thereof,
in the presence of a suitable base and a suitable solvent,
to form the compound of formula Ia;
and optionally converting the compound of formula Ia to a salt thereof.

In another aspect, the invention provides a pharmaceutical composition comprising a compound of the invention and a pharmaceutically acceptable carrier. The pharmaceutical composition can be formulated for particular routes of administration such as oral administration, parenteral administration, and rectal administration, etc. In addition, the pharmaceutical compositions of the invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The invention further provides anhydrous pharmaceutical compositions and dosage forms comprising the compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the invention as an active ingredient will decompose. Such agents, which are referred to herein as “stabilizers,” include, but are not limited to, antioxidants such as ascorbic acid, pH buffers, or salt buffers, etc.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of formula I or pharmaceutical acceptable salts thereof exhibit valuable pharmacological properties and are therefore useful as pharmaceuticals. Furthermore, compounds of formula I may be useful for research on mGluR5, e.g. as tool compounds.

In particular, compounds of formula I exhibit an antagonistic action at human metabotropic glutamate receptor 5 (human mGluR5). This can be determined in vitro, for example, at recombinant human mGluR5, using different procedures like, for example, measurement of the inhibition of the agonist induced elevation of intracellular Ca²⁺ concentration in accordance with L. P. Daggett et al., Neuropharm. Vol. 34, pages 871-886 (1995), P. J. Flor et al., J. Neurochem. Vol. 67, pages 58-63 (1996) or by determination to what extent the agonist induced elevation of the inositol phosphate turnover is inhibited as described by T. Knoepfel et al., Eur. J. Pharmacol. Vol. 288, pages 389-392 (1994), L. P. Daggett et al., Neuropharm. Vol. 67, pages 58-63 (1996) and references cited therein. Isolation and expression of human mGluR subtypes are described in U.S. Pat. No. 5,521,297.

Selected compounds of formula (I) show IC₅₀ values for the inhibition of the agonist (e.g. glutamate or quisqualate) induced elevation of intracellular Ca2+ concentration or the agonist (e.g. glutamate or quisqualate) induced inositol phosphate turnover, measured in recombinant cells expressing hmGluR5a of about 1 nM to about 10 μM.

Preferred compounds of formula (I) show an inhibition of said inositol phosphate turnover in recombinant cells expressing hmGluR5a of at least 1 μM.

Further preferred compounds of formula (I) show an IC₅₀ value of said inositol phosphate turnover in recombinant cells expressing hmGluR5a of at least 500 nM.

Further preferred compounds of formula (I) show an IC₅₀ value of said inositol phosphate turnover in recombinant cells expressing hmGluR5a of at least 250 nM.

Further preferred compounds of formula (I) show an IC₅₀ value of said inositol phosphate turnover in recombinant cells expressing hmGluR5a of at least 100 nM.

The compounds of the invention may be therefore useful in the prevention, treatment or delay of progression of disorders associated with irregularities of the glutamatergic signal transmission, of the gastro-intestinal and urinary tract and of nervous system disorders mediated full or in part by mGluR5.

Disorders associated with irregularities of the glutamatergic signal transmission are for example epileptogenesis including neuronal protection after status epilepticus, cerebral ischemias, especially acute ischemias, ischemic diseases of the eye, muscle spasms such as local or general spasticity, skin disorders, obesity disorders and, in particular, convulsions or pain.

Disorders of the gastro-intestinal tract include Gastro-Esophageal Reflux Disease (GERD), Functional Gastro-intestinal Disorders and Post-operative Ileus.

Functional Gastro-intestinal Disorders (FGIDs) are defined as chronic or recurrent conditions associated with abdominal symptoms without organic cause using conventional diagnostic measures. A cardinal symptom present in many FGIDs is visceral pain and/or discomfort. FGIDs include functional dyspepsia (FD), functional heartburn (a subset of GERD), irritable bowel syndrome (IBS), functional bloating, functional diarrhea, chronic constipation, functional disturbancies of the biliary tract as well as other conditions according to Gut 1999; Vol. 45 Suppl. II. A disorder of particular interest is GERD.

Post-operative Ileus is defined as failure of aboral passage of intestinal contents due to transient impairment of GI motility following abdominal surgery.

Disorders of the Urinary Tract comprise conditions associated with functional disturbancies and/or discomfort/pain of the urinary tract. Examples of disorders of the urinary tract include but are not limited to incontinence, benign prostatic hyperplasia, prostatitis, detrusor hyperreflexia, outlet obstruction, urinary frequency, nocturia, urinary urgency, overactive bladder (OAB), pelvic hypersensitivity, urge incontinence, urethritis, prostatodynia, cystitis, idiopathic bladder hypersensitivity and the like. OAB is a syndrome characterized by urgency, with or without urinary incontinence, and usually with increased voiding frequency and nocturia.

Nervous system disorders mediated full or in part by mGluR5 are for example acute, traumatic and chronic degenerative processes of the nervous system, such as Parkinson's disease, Parkinson's dyskinesia (e.g. L-dopa induced dyskinesia), dyskinesias induced by neuroleptics (e.g. tardive dyskenisia), Tic disorders, Tourette Syndrome, Restless Leg Syndrome, Periodic Limb Movement Syndromes, senile dementia, Alzheimer's disease, Huntington's chorea, amyotrophic lateral sclerosis, multiple sclerosis and fragile X syndrome, substance-related disorders, psychiatric diseases such as schizophrenia, affective and anxiety disorders, attention deficit disorders and cognitive dysfunction associated with these and other CNS disorders. Substance-related disorders include substance abuse, substance dependence and substance withdrawal disorders, e.g. nicotine withdrawal. Anxiety disorders includes panic disorder, social and specific phobias, anxiety, obsessive compulsive disorder (OCD), post traumatic stress disorder (PTSD) and generalized anxiety disorder (GAD). Affective disorders include depressive (major depression, dysthymia, depressive disorders NOS) and bipolar disorders (bipolar I and II disorders). Cognitive dysfunction associated with these and other CNS disorders include deficits and abnormalities in attention and vigilance, executive functions and memory (for instance working memory and episodic memory). Other disorders which are mediated fully or in part by mGluR5 are pain and itch.

A disorder of particular interest is L-dopa induced dyskinesia in Parkinsons Disease.

The compounds of the invention, especially the compounds as defined in group P under Embodiment 8, are useful in the treatment, prevention or delay of progression of dyskinesias in Parkinsons Disease, especially L-dopa induced dyskinesia in Parkinsons Disease. Dyskinesia in Parkinsons Disease often, although not exclusively, occurs as a side-effect of treatment of Parkinson's Disease with levodopa (L-dopa), a precursor of dopamine. Characteristics of such dyskinesia include motor impairment, e.g. the appearance of slow and uncoordinated involuntary movements, shaking, stiffness and problems walking. Patients treated with L-dopa often have reduced symptoms of Parkinson's Disease but they experience increasing difficulties to remain standing or even sitting. After prolonged use of L-dopa, a majority of patients develop dyskinesia.

Dyskinesia can occur at any time during the cycle of treatment with L-dopa. In one embodiment, the compounds of the invention are for the treatment of dyskinesia which occurs at the time of peak L-dopa plasma concentrations in the patient. In one embodiment, the compounds of the invention are for the treatment of dyskinesia which occurs when the L-dopa plasma concentrations in a patient rise or fall (diphasic dyskinesia).

Dyskinesia can also develop in Parkinson's disease sufferers who do not take L-dopa. In one embodiment, the compounds of the invention are for the treatment of non-L-dopa induced Parkinson's dyskinesia.

Treatment with a compound of the invention, especially with a compound as defined in group P, may comprise a reduction in the characteristics associated with Parkinson's dyskinesia, including for example, although not limited to, a reduction in the scale of involuntary movements, a reduction in the number of involuntary movements, an improvement in the ability to carry out normal tasks, an improved ability to walk, increased period of time between episodes of dyskinesia.

In the case of prophylactic treatment, the compounds of the invention, especially the compounds as defined in group P may be used to delay or prevent the onset of Parkinson's dyskinesia.

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 0.1 to about 10 mg/kg body weight, e.g. 1 mg/kg. In larger mammals, for example humans, an indicated daily dosage is in the range from about 0.1 to about 1000 mg, preferably from about 1 to about 400 mg, most preferably from about 10 to about 100 mg of the compound of the invention conveniently administered, for example, in divided doses up to four times a day.

For use according to the invention, a compound of the invention, especially a compound as defined in group P, may be administered as single active agent or in combination with other active agents, in any usual manner, e.g. orally, for example in the form of tablets or capsules, or parenterally, for example in the form of injection solutions or suspensions. A combination comprising a compound of the invention and another active agent will be referred to as “combination of the invention”.

In the case of Parkinson's dyskinesia induced by L-dopa, the compound of the invention, especially being a compound as defined in group P, will be combined with L-dopa and optionally with at least one active agent selected from the group consisting of a dopa decarboxylase inhibitor, a catechol-O-methyl transferase inhibitor, a dopamine agonist, a monoamine oxidase-B inhibitor, an adrenergic drug, a drug for obstructed airway disorders, a beta blocking agent, an alpha-adrenoreceptor antagonist, an angiotensin II antagonist, an anticholinergic, an anticholinesterase, an antidepressant, an anti-inflammatory agent, an anti-rheumatic agent, an antimigraine agent, an anxiolytic, a barbiturate, a barbiturate derivate, a belladonna alkaloid, a tertiary amine and a benzothiazepine derivative.

Dopa decarboxylase inhibitors are, for example, carbidopa or benserazide. Catechol-O-methyl transferase inhibitors are, for example, tolcapone or entacapone.

Dopamine agonists are, for example, bromocriptine, pergolide, pramipexole, ropinirole, cabergoline, apomorphine or lisuride.

Monoamine oxidase-B inhibitors are, for example, selegiline, rasagiline.

Adrenergics and/or drugs for obstructed airway disorders are, for example, Budesonide with formoterol fumarate, Combivent, Sertide mite or Salbutamol.

Beta blocking agents are, for example, Acebutolol, Acebutolol hydrochloride, Atenolol, Betaxolol, Betaxolol hydrochloride, Bisoprolol, Bisoprolol fumarate, Bisoprolol hemifumarate, Carvedilol, Cosopt, Levobunolol hydrochloride, Metoprolol, Metoprolol succinate, Metoprolol tartrate, Propranolol, Propranolol hydrochloride, Sotalol, Sotalol hydrochloride, Tenoretic, Timolol, Timolol maleate or Timpilo.

Alpha-adrenoreceptor antagonists are, for example, Alfuzosin, Alfuzosin hydrochloride, Doxazosin, Doxazosin mesilate, Tamsulosin, Tamsulosin hydrochloride, Terazosin or Terazosin hydrochloride.

Angiotensin II antagonists are, for example, Candesartan cilexetil, Irbesartan, Losartan, Losartan potassium, Olmesartan medoxomil, Telmisartan or Valsartan.

Combinations of Angiotensin II antagonists are, for example, Blopress plus, Co-diovan, Hyzaar or Karvea hct.

Anticholinergics are, for example, Ibratropium bromide or Tiotropium bromide.

Anticholinesterases are, for example, Donepezil hydrochloride.

Antidepressants are, for example, Amitriptyline, Amitriptyline hydrochloride, Bupropion hydrochloride, Citalopram, Citalopram hydrobromide, Cyclobenzaprine, Cyclobenzaprine hydrochloride, Escitalopram, Escitalopram oxalate, Fluoxetine, Fluvoxamine maleate, Imipramine hydrochloride, Mirtazapine, Paroxetine, Paroxetine hydrochloride, Sertraline, Sertraline hydrochloride, Trazodone, Trazodone hydrochloride, Venlafaxine or Venlafaxine hydrochloride.

Antiepileptics are, for example, Carbamazepine, Clonazepam, Gabapentin, Phenobarbital, Phenyloin, Pregabalin or Topiramate.

Anti-inflammatory and/or anti-rheumatic agents are, for example, Betamethasone, Betamethasone valerate, Cortisone, Cortisone acetate, Desonide, Diclofenac, Diclofenac sodium, Flurbiprofen, Hydrocortisone, Indometacin, Salicylic acid, Triamcinolone acetonide, Aceclofenac, Aflexa, Arthrotec, Carbager-plus, Celecoxib, Glucosamine, Glucosamine sulfate, Glucosamine with chondroitin, Ibuprofen, Ketoprofen, Meloxicam, Naproxen, Naproxen sodium, Nimesulide, Osteo bi-flex or Sulindac.

Antimigraine preparations are, for example, Naratriptan hydrochloride, Rizatriptan or Sumatriptan.

Anxiolytics are, for example, Alprazolam, Bromazepam, Clonazepam, Clorazepate dipotassium, Diazepam, Ethyl loflazepate, Hydroxyzine, Hydroxyzine hydrochloride, Lorazepam, Oxazepam or Tetrazepam.

Barbiturates and/or barbiturate derivates are, for example, Phenobarbital or Phenobarbital. Belladonna alkaloids and/or tertiary amines are, for example, Hyoscyamine sulfate Benzodiazepine derivatives and related drugs are, for example, Alprazolam, Bromazepam, Clonazepam, Clorazepate dipotassium, Diazepam, Ethyl loflazepate, Lorazepam, Lormetazepam, Oxazepam, Temazepam, Tetrazepam, Triazolam, Eszopiclone, Zolpidem, Zolpidem tartrate or Zopiclone.

Benzothiazepine derivatives are, for example, Diltiazem or Diltriazem hydrochloride.

In one embodiment of the invention a specific combination of the invention is used. Said combination comprises:

A compound of the invention, especially a compound as defined in group P; and L-dopa.

In one embodiment of the invention a specific combination of the invention is used. Said combination comprises:

A compound of the invention, especially a compound as defined in group P;

### L-dopa; and

at least one active agent selected from the group consisting of:
carbidopa, benserazide, tolcapone, entacapone, bromocriptine, pergolide, pramipexole, ropinirole, cabergoline, apomorphine, lisuride, selegiline, rasagiline, Budesonide with formoterol fumarate, Combivent, Sertide mite, Salbutamol, Acebutolol, Acebutolol hydrochloride, Atenolol, Betaxolol, Betaxolol hydrochloride, Bisoprolol, Bisoprolol fumarate, Bisoprolol hemifumarate, Carvedilol, Cosopt, Levobunolol hydrochloride, Metoprolol, Metoprolol succinate, Metoprolol tartrate, Propranolol, Propranolol hydrochloride, Sotalol, Sotalol hydrochloride, Tenoretic, Timolol, Timolol maleate, Timpilo, Alfuzosin, Alfuzosin hydrochloride, Doxazosin, Doxazosin mesilate, Tamsulosin, Tamsulosin hydrochloride, Terazosin, Terazosin hydrochloride, Candesartan cilexetil, Irbesartan, Losartan, Losartan potassium, Olmesartan medoxomil, Telmisartan, Valsartan, Blopress plus, Co-diovan, Hyzaar, Karvea hct, Ibratropium bromide, Tiotropium bromide, Donepezil hydrochloride, Amitriptyline, Amitriptyline hydrochloride, Bupropion hydrochloride, Citalopram, Citalopram hydrobromide, Cyclobenzaprine, Cyclobenzaprine hydrochloride, Escitalopram, Escitalopram oxalate, Fluoxetine, Fluvoxamine maleate, Imipramine hydrochloride, Mirtazapine, Paroxetine, Paroxetine hydrochloride, Sertraline, Sertraline hydrochloride, Trazodone, Trazodone hydrochloride, Venlafaxine, Venlafaxine hydrochloride, Carbamazepine, Clonazepam, Gabapentin, Phenobarbital, Phenyloin, Pregabalin, Topiramate, Betamethasone, Betamethasone valerate, Cortisone, Cortisone acetate, Desonide, Diclofenac, Diclofenac sodium, Flurbiprofen, Hydrocortisone, Indometacin, Salicylic acid, Triamcinolone acetonide, Aceclofenac, Aflexa, Arthrotec, Carbager-plus, Celecoxib, Glucosamine, Glucosamine sulfate, Glucosamine with chondroitin, Ibuprofen, Ketoprofen, Meloxicam, Naproxen, Naproxen sodium, Nimesulide, Osteo bi-flex or Sulindac. Antimigraine preparations are, for example, Naratriptan hydrochloride, Rizatriptan, Sumatriptan, Alprazolam, Bromazepam, Clonazepam, Clorazepate dipotassium, Diazepam, Ethyl loflazepate, Hydroxyzine, Hydroxyzine hydrochloride, Lorazepam, Oxazepam, Tetrazepam, Phenobarbital, Phenobarbital, Hyoscyamine sulfate, Alprazolam, Bromazepam, Clonazepam, Clorazepate dipotassium, Diazepam, Ethyl loflazepate, Lorazepam, Lormetazepam, Oxazepam, Temazepam, Tetrazepam, Triazolam, Eszopiclone, Zolpidem, Zolpidem tartrate, Zopiclone, Diltiazem and Diltriazem hydrochloride.

An example of a combination is a compound as defined in group P, L-dopa, and the dopa decarboxylase inhibitor carbidopa.

Another example of a combination is a compound as defined in group P, L-dopa, and entacapone.

Another example of a combination is a compound as defined in group P, L-dopa, entacapone, and carbidopa; an example of such a combination is a combination of a compound as defined in group P and Stalevo®.

An example of a combination is the first compound as defined in group P, i.e. (3aR,4S,7aR)-methyl 4-((3-chlorophenyl)ethynyl)-4-hydroxyoctahydro-1H-indole-1-carboxylate, L-dopa, and the dopa decarboxylase inhibitor carbidopa.

Another example of a combination is the first compound as defined in group P, i.e. (3aR,4S,7aR)-methyl 4-((3-chlorophenyl)ethynyl)-4-hydroxyoctahydro-1H-indole-1-carboxylate, L-dopa, and entacapone.

Another example of a combination is the first compound as defined in group P, i.e. (3aR,4S,7aR)-methyl 4-((3-chlorophenyl)ethynyl)-4-hydroxyoctahydro-1H-indole-1-carboxylate, L-dopa, entacapone, and carbidopa; an example of such a combination is a combination of the first compound as defined in group P, i.e. (3aR,4S,7aR)-methyl 4-((3-chlorophenyl)ethynyl)-4-hydroxyoctahydro-1H-indole-1-carboxylate, and Stalevo®.

The agents of the present invention may also be useful for treating or preventing migraine.

The agents of the present invention may also be useful for inflammatory diseases, such as pain, inflammation and/or oedema consequential to trauma, for example associated with burns, sprains, fractures or the like, inflammatory airways diseases, such as COPD, asthma, rhinitis, inflammatory bowel disease, cystitis, uveitis, inflammatory skin disorders, such as psoriasis or eczema, rheumatoid arthritis, use as a smooth muscle relaxant, for example for the treatment of spasms of the gastro-intestinal tract or uterus, for example in the therapy of Crohn's disease, ulcerative collitis or pancreatitis, or for the treatment of muscle spasticity and tremor, for example in multiple sclerosis, teno-synovitis, gout, ocular disorders, for example glaucoma, cough.

The agents of the present invention may also be useful for treating cognitive impairment and/or attention deficit disorder.

Cognitive dysfunction include deficits and abnormalities in attention and vigilance, executive functions and memory (for instance working memory and episodic memory). Other disorders relating to cognitive dysfunction include sleep related breathing disorders (SRBD), behavioral impairments, information processing deficits and age-related disorders. Further examples falling of cognitive impairment and/or attention deficit disorders include: Attention-deficit hyperactivity disorder (ADHD), childhood ADHD, adult ADHD, excess daytime somnolence, sleep apnea, shift-worker's sleep-wake cycle disruption, traumatic brain injury, neurodegenerative disorders with associated memory and cognitive problems (such as Alzheimer's disease, Lewy body dementia, senile dementia, vascular dementia, Parkinson's disease), chronic fatigue syndrome, fatigue associated with sleep deprivation or prolonged wakefulness, age-related decline in memory and cognitive function (such as mild cognitive impairment), cognitive impairment associated with mood disorders (such as depression) and anxiety, schizophrenia, day time sleepiness associated with narcolepsy.

Furthermore, the agents of the present invention may provide treatment for or improve of the cognitive enhancement of a subject. The term “cognitive enhancement” includes, but is not limited to, cognition enhancement, vigilance, counteracting effects of fatigue, enhancing alertness, attention, memory (working, episodic), learning ability, reaction time, cognitive performance enhancement, excess daytime somnolence, reversal of information processing deficits, improvement of disorganization, i.e. improving organizational skills/level of organizational ability.

The agents of the present invention may also be useful for treating pervasive developmental disorders (PDD). PDD is a group of diseases characterized by a delay in the development of socialization and communications skills. The following diseases are part of the PDD: Autism, Asperger's syndrome, childhood disintegrative disorder, and Rett's syndrome, and fragile X. The main symptomotology are: Autistic-like behavior, repetitive behavior (OCD), in some cases irritability, and ADHS. Fragile X Syndrome have two different genotype-phenotype: Full mutation (mental retardation, ADHD, autism, and anxiety), partial mutation (tremor-ataxia, parkinsonism, anxiety). A disorder of particular interest is fragile X syndrome.

The compounds of the invention may be useful for the prevention of the above-mentioned conditions and disorders.

The compounds of the invention may be useful for the treatment of the above-mentioned conditions and disorders.

The compounds of the invention may be useful for the delay of progression of the above-mentioned conditions and disorders.

Compounds of the invention may be especially useful in the treatment of an indication selected from: L-dopa induced dyskinesias in Parkinsons Disease and fragile X syndrome.

Thus, as a further embodiment, the invention provides the use of a compound of formula (I) or a pharmaceutically acceptable salt thereof as a medicament.

As a further embodiment, the invention provides the use of a compound of formula (I) or a pharmaceutically acceptable salt thereof in therapy.

In a further embodiment, the therapy is selected from a disease which is ameliorated by inhibition of mGluR5. In another embodiment, the disease is selected from the afore-mentioned list, e.g. L-dopa induced dyskinesias in Parkinsons Disease and fragile X syndrome.

In another embodiment, the invention provides a method of treating a disease which is ameliorated by inhibition of mGluR5 comprising administration of a therapeutically acceptable amount of a compound of formula (I) or a pharmaceutically acceptable salt thereof. In a further embodiment, the disease is selected from the afore-mentioned list, suitably L-dopa induced dyskinesias in Parkinsons Disease and fragile X syndrome.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by mGluR5, or (ii) associated with mGluR5 activity, or (iii) characterized by abnormal activity of mGluR5; or (2) reducing or inhibiting the activity of mGluR5; or (3) reducing or inhibiting the expression of mGluR5. In another non-limiting embodiment, the term “a therapeutically effective amount” refers to the amount of the compound of the invention that, when administered to a cell, or a tissue, or a non-cellular biological material, or a medium, is effective to at least partially reducing or inhibiting the activity of mGluR5; or at least partially reducing or inhibiting the expression of mGluR5.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The pharmaceutical composition or combination of the invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound of the invention can be assessed by in vitro & in vivo methods described herein.

The compound of the invention may be administered either simultaneously with, or before or after, at least one other therapeutic agent. The compound of the invention may be administered separately, by the same or different route of administration, or together in the same pharmaceutical composition.

The following Examples illustrate the invention, but do not limit it.

### EXPERIMENTAL PART

### General

Analytical UPLC/MS conditions (%=percent by volume): Waters Acquity UPLC system, column Acquity HSS-T3 1.8 μm; 2.1×50 mm; T=50° C.; gradient: A, water+0.05% HCO2H+0.05% ammonium acetate; B, acetonitrile+0.4% HCO₂H; from A/B 98/2 to 2/98 in 1.4 min+0.57 min isocratic; flow rate 1.2 ml/min.

MS detector: Waters Acquity SQD (Single Quadrupol Detector).

1H NMR spectra were acquired on a Bruker spectrometer (400 MHz). Chemical shifts are given in parts per million (ppm) relative to the residual solvent peak.

### ABBREVIATIONS

AcOEt ethyl acetate
aq. aqueous
BuLi butyl lithium
DMF dimethylformamide
h hour
M molar
MeOH methanol
mg milligram
min minute
mL milliliter
μL microliter
mmol millimole
MS mass spectrometry
org. organic
rt retention time
RT room temperature
sat. saturated
soln. solution
THF tetrahydrofuran
UPLC ultra pressure liquid chromatography

### Intermediates

### Intermediate A: 3-ethynyl-5-methylpyridine

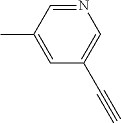

Step A: A suspension of 3-bromo-5-methylpyridine (250 mg, 1.45 mmol), ethynyltrimethylsilane (257 mg. 2.62 mmol), Et₃N (403 μL, 2.91 mmol), Pd(PPh₃)₄ (84 mg, 0.07 mmol) and CuI (28 mg, 0.15 mmol) in dry DMF (2 mL) was heated to 140° C. for 7 h. The mixture was then poured onto H₂O (100 mL) and extracted with AcOEt (2*100 mL). The combined org. phases were dried over Na₂SO₄, filtered and concentrated in vacuo. The crude product was purified by flash chromatography (SiO₂, Heptane/AcOEt 100:0 to 60:40) to provide 3-methyl-5-((trimethylsilyl)ethynyl)pyridine (164 mg, 60% yield). UPLC/MS: rt=1.23 min; found 190.2 [M+H⁺].

Step B: A soln. of 3-methyl-5-((trimethylsilyl)ethynyl)pyridine (161 mg, 0.85 mmol) in MeOH (10 mL) was treated with K₂CO₃ (235 mg, 1.70 mmol) and stirred at RT for 1 h. The mixture was then concentrated in vacuo and the crude product was purified by flash chromatography (SiO₂, Heptane/AcOEt 100:0 to 0:100) to give 3-ethynyl-5-methylpyridine (45 mg, 45% yield). UPLC/MS: rt=0.70 min, found 118.1 [M+H⁺]. ¹H-NMR (400 MHz, d₆-DMSO): 8.46 (s, 1H); 8.41 (s, 1H); 7.72 (s, 1H); 4.39 (s, 1H); 2.28 (s, 3H).

### Intermediate B: (3aR,4S,7aR)-methyl 4-ethynyl-4-hydroxyoctahydro-1H-indole-1-carboxylate

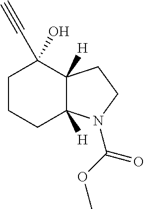 [SMILES: C#C[C@]1(O)CCC[C@@H]2[C@H]1CCN2C(=O)OC]

Step A: A solution of ethynyltrimethylsilane (8.22 g, 84 mmol) in anhydrous THF (300 mL) was cooled to −20° C., treated dropwise with BuLi (1.6 M in hexane, 66.5 mL, 106 mmol), stirred for 2 h at this temperature, and then cooled to −78° C. A solution of (3aR,7aR)-methyl 4-oxooctahydro-1H-indole-1-carboxylate (15.0 g, 76 mmol) in anhydrous THF (50 mL) was then added dropwise over 10 min, and then allowed to warm to 0° C. over 1 h, and stirred for 1 h at this temperature. The reaction mixture was poured onto a sat. soln. of NaHCO₃ (500 mL) and extracted with AcOEt (2*300 mL). The combined org. layers were then dried over Na₂SO₄, filtered and concentrated in vacuo to provide (3aR,4S,7aR)-methyl 4-hydroxy-4-((trimethylsilyl)ethynyl)octahydro-1H-indole-1-carboxylate (20 g, 89% crude yield), which was used as it is in the next step. Crude product: ¹H-NMR (400 MHz, d₆-DMSO): 5.61 (s, 1H); 3.67-3.75 (m, 1H); 3.54 (s, 3H); 3.32-3.39 (m, 1H); 3.19-3.28 (m, 1H); 2.37-2.45 (m, 1H); 1.42-1.84 (m, 7H); 0.99-1.07 (m, 1H); 0.13 (s, 9H).

Step B: A solution of (3aR,4S,7aR)-methyl 4-hydroxy-4-((trimethylsilyl)ethynyl)octahydro-1H-indole-1-carboxylate (20 g, 68 mmol) in MeOH (200 mL) was treated with K₂CO₃ (18.7 g, 135 mmol) and stirred for 1 h at RT. The reaction mixture was then concentrated in vacuo, dissolved in H₂O (400 mL), and extracted with AcOEt (2*200 mL). The combined org. phases were dried over Na₂SO₄, filtered and concentrated in vacuo to give (3aR,4S,7aR)-methyl 4-ethynyl-4-hydroxyoctahydro-1H-indole-1-carboxylate (14.3 g, 95% yield). UPLC/MS: rt=no UV trace; found 224.3 [M+H⁺]. ¹H-NMR (400 MHz, d₆-DMSO): 5.60 (s, 1H); 3.70-3.78 (m, 1H); 3.53 (s, 3H); 3.30-3.37 (m, 1H), 3.30 (s, 1H); 3.18-3.29 (m, 1H); 2.39-2.46 (m, 1H); 1.37-1.86 (m, 7H); 1.00-1.10 (m, 1H).

### Intermediate C: (3aR,7aR)-methyl 4-(4-methoxybenzylamino)-4-(m-tolylethynyl)octahydro-1H-indole-1-carboxylate

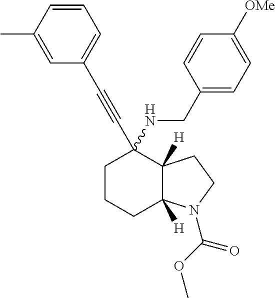 [SMILES: COC(=O)N1CC[C@@H]2[C@H]1CCCC2(C#Cc1cccc(C)c1)NCc1ccc(OC)cc1]

Step A: A solution of (3aR,7aR)-methyl 4-oxooctahydro-1H-indole-1-carboxylate (2.00 g, 10.1 mmol) in toluene (10 mL) was treated with 4-methoxybenzylamine (1.53 g, 11.2 mmol) and BF₃.OEt₂ (129 μL, 1.01 mmol), and the mixture was heated to 120° C. for 4 h. The mixture was then poured onto a sat. aq. soln. of NaHCO₃ (300 mL) and extracted with CH₂Cl₂ (2*250 mL). The combined org. phases were dried over Na₂SO₄, filtered and concentrated in vacuo to give (3aR,7aR,Z)-methyl 4-(4-methoxybenzylimino)octahydro-1H-indole-1-carboxylate (3.1 g, 97% crude yield), which was used as it is in the next step.

Step B: A solution of 1-ethynyl-3-methyl-benzene (1.14 g, 9.80 mmol) in anhydrous THF (80 mL) was cooled to −78° C., treated dropwise over 2 min with BuLi (1.6 M in hexane, 7.35 mL, 11.8 mmol), and allowed to slowly warm to −20° C. over 1 h. Another solution of crude (3aR,7aR,Z)-methyl 4-(4-methoxybenzylimino)octahydro-1H-indole-1-carboxylate (3.1 g, 9.80 mmol) in anhydrous THF (50 mL) was cooled to −78° C., treated with BF₃.OEt₂ (1.24 mL, 19.80 mmol), and then rapidly added to the first solution at −20° C. The mixture was allowed to warm to RT, stirred for 1 h, and then poured onto a sat. aq. soln. of NaHCO₃ (400 mL) and extracted with AcOEt (2*250 mL). The combined org. phases were dried over Na₂SO₄, filtered and concentrated in vacuo. Purification by flash chromatography (SiO₂, Heptane/AcOEt 80:20) to afford (3aR,7aR)-methyl 4-(4-methoxybenzylamino)-4-(m-tolylethynyl)octahydro-1H-indole-1-carboxylate (800 mg, 19% yield). UPLC/MS: rt=1.03 min; found 433.5 [M+H⁺].

### Final Products

### Example 1

### (3aR,4S,7aR)-methyl 4-((3-chlorophenyl)ethynyl)-4-hydroxyoctahydro-1H-indole-1-carboxylate

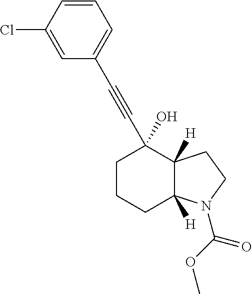 [SMILES: COC(=O)N1CC[C@@H]2[C@H]1CCC[C@@]2(O)C#Cc1cccc(Cl)c1]

A solution of 1-chloro-3-ethynylbenzene (450 mg, 3.30 mmol) in anhydrous THF (60 mL) was cooled to −20° C., treated dropwise with BuLi (1.6 M in hexane, 2.06 mL, 3.30 mmol), stirred for 2 h at this temperature, and cooled to −78° C. A solution of (3aR,7aR)-methyl 4-oxooctahydro-1H-indole-1-carboxylate (500 mg, 2.54 mmol) in anhydrous THF (10 mL) was then added dropwise over 3 min, and the mixture was then allowed to warm to RT. The reaction mixture was poured onto a sat. soln. of NaHCO₃ (200 mL) and extracted with AcOEt (2*200 mL). The combined org. phases were dried over Na₂SO₄, filtered and concentrated in vacuo. The crude product was purified by flash chromatography (SiO₂, Heptane/AcOEt/MeOH 100:0:0 to 0:90:10 and CH₂Cl₂/MeOH 100:0 to 80:20) followed by recrystallization in heptane to provide (3aR,4S,7aR)-methyl 4-((3-chlorophenyl)ethynyl)-4-hydroxyoctahydro-1H-indole-1-carboxylate (290 mg, 34% yield). UPLC/MS: rt=1.06 min; found 334.2/336.2 [M+H⁺]. ¹H-NMR (400 MHz, d₆-DMSO): 7.37-7.48 (m, 4H); 5.77 (s, 1H); 3.73-3.89 (m, 1H); 3.54 (s, 3H); 3.32-3.45 (m, 1H); 3.20-3.29 (m, 1H); 2.51-2.59 (m, 1H); 1.52-1.88 (m, 7H); 1.04-1.14 (m, 1H).

### Example 2

### (3aR,4S,7aR)-methyl 4-hydroxy-4-((5-methylpyridin-3-yl)ethynyl)octahydro-1H-indole-1-carboxylate

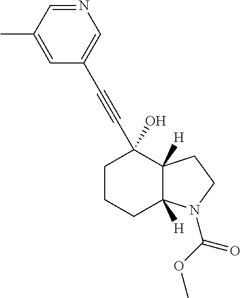 [SMILES: COC(=O)N1CC[C@@H]2[C@H]1CCC[C@@]2(O)C#Cc1cncc(C)c1]

Example 2 can be prepared by analogy to Example 1, using intermediate A. UPLC/MS: rt=0.80 min; found 315.3 [M+H⁺]. ¹H-NMR (400 MHz, CD₃OD): 8.39 (s, 1H); 8.34 (s, 1H); 7.72 (s, 1H); 3.95-4.03 (m, 1H); 3.67 (s, 3H); 3.49-3.56 (m, 1H); 3.31-3.42 (m, 1H); 2.65-2.74 (m, 1H); 2.35 (s, 3H); 1.64-2.06 (m, 7H); 1.17-1.25 (m, 1H).

### Example 3

### (3aR,4S,7aR)-methyl 4-hydroxy-4-((2-methylpyridin-4-yl)ethynyl)octahydro-1H-indole-1-carboxylate

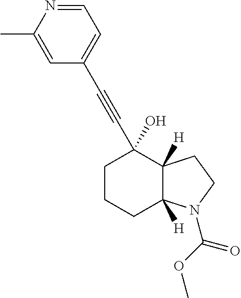 [SMILES: COC(=O)N1CC[C@@H]2[C@H]1CCC[C@@]2(O)C#Cc1ccnc(C)c1]

A solution of (3aR,4S,7aR)-methyl 4-ethynyl-4-hydroxyoctahydro-1H-indole-1-carboxylate (intermediate B, 100 mg, 0.45 mmol), 4-bromo-2-methylpyridine (77 mg, 0.45 mmol), Et₃N (94 μL, 0.67 mmol), CuI (4.3 mg, 0.022 mmol), and Pd(PPh₃)₄ (5.2 mg, 0.005 mmol) in anhydrous DMF (5 mL) was degased and heated to 140° C. for 20 h under Ar. The mixture was then diluted with AcOEt, filtered over Hyflo, and the filtrate was concentrated in vacuo The crude product was purified by flash chromatography (SiO₂, heptane/AcOEt 100:0 to 0:100), and the product was recrystallized in heptane to give (3aR,4S,7aR)-methyl 4-hydroxy-4-((2-methylpyridin-4-yl)ethynyl)octahydro-1H-indole-1-carboxylate (10 mg, 7% yield). UPLC/MS: rt=0.65 min; found 315.3 [M+H⁺]. ¹H-NMR (400 MHz, d₆-DMSO): 8.41 (d, J=5.0 Hz, 1H); 7.27 (s, 1H); 7.17 (d, J=5.1 Hz, 1H); 5.85 (s, 1H); 3.76-3.83 (m, 1H); 3.53 (s, 3H); 3.34-3.39 (m, 1H); 3.22-3.28 (m, 1H); 2.53-2.58 (m, 1H); 2.43 (s, 3H); 1.46-1.88 (m, 7H); 1.05-1.12 (m, 1H).

Examples 4-7 can be prepared by analogy, using intermediate B and the appropriate brominated heterocycle.

### Example 4

### (3aR,4S,7aR)-methyl 4-hydroxy-4-((6-methylpyridin-2-yl)ethynyl)octahydro-1H-indole-1-carboxylate

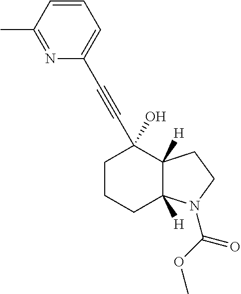 [SMILES: COC(=O)N1CC[C@@H]2[C@H]1CCC[C@@]2(O)C#Cc1cccc(C)n1]

UPLC/MS: rt=0.77 min; found 315.0 [M+H⁺]. ¹H-NMR (400 MHz, d₆-DMSO): 7.66 (t, J=7.8 Hz, 1H); 7.28 (d, J=7.8 Hz, 1H); 7.22 (d, J=7.9 Hz, 1H); 5.83 (s, 1H), 3.78-3.85 (m, 1H); 3.54 (s, 3H); 3.36-3.42 (m, 1H); 3.23-3.30 (m, 1H); 2.51-2.59 (m, 1H); 2.43 (s, 3H); 1.48-1.89 (m, 7H); 1.08-1.15 (m, 1H).

### Example 5

### (3aR,4S,7aR)-methyl 4-((2-chloropyridin-4-yl)ethynyl)-4-hydroxyoctahydro-1H-indole-1-carboxylate

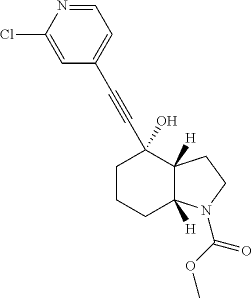 [SMILES: COC(=O)N1CC[C@@H]2[C@H]1CCC[C@@]2(O)C#Cc1ccnc(Cl)c1]

UPLC/MS: rt=0.89 min; found 335.3 [M+H⁺]. ¹H-NMR (400 MHz, d₆-DMSO): 8.39 (d, J=5.1 Hz, 1H); 7.59 (d, J=3.2 Hz, 1H); 7.42 (d, J=5.1 Hz, 1H); 5.91 (s, 1H); 3.79-3.85 (m, 1H); 3.53 (s, 3H); 3.34-3.40 (m, 1H); 3.21-3.29 (m, 1H); 2.52-2.58 (m, 1H); 1.53-1.90 (m, 7H); 1.01-1.11 (m, 1H).

### Example 6

### (3aR,4S,7aR)-methyl 4-hydroxy-4-((4-methylpyridin-2-yl)ethynyl)octahydro-1H-indole-1-carboxylate

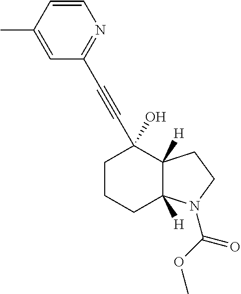 [SMILES: COC(=O)N1CC[C@@H]2[C@H]1CCC[C@@]2(O)C#Cc1cc(C)ccn1]

UPLC/MS: rt=0.79 min; found 315.4 [M+H⁺]. ¹H-NMR (400 MHz, d₆-DMSO): 8.37 (d, J=5.1 Hz, 1H); 7.33 (s, 1H); 7.19 (d, J=5.0 Hz, 1H); 5.83 (s, 1H); 3.76-3.86 (m, 1H); 3.54 (s, 3H); 3.34-3.42 (m, 1H); 3.21-3.29 (m, 1H); 2.51-2.58 (m, 1H); 2.29 (s, 3H); 1.49-1.89 (m, 7H); 1.05-1.17 (m, 1H).

### Example 7

### (3aR,4S,7aR)-methyl 4-hydroxy-4-((2-methylthiazol-4-yl)ethynyl)octahydro-1H-indole-1-carboxylate

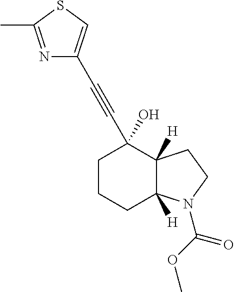 [SMILES: COC(=O)N1CC[C@@H]2[C@H]1CCC[C@@]2(O)C#Cc1csc(C)n1]

UPLC/MS: rt=0.78 min; found 321.1 [M+H⁺]. ¹H-NMR (400 MHz, d₆-DMSO): 7.74 (s, 1H); 5.80 (d, J=3.1 Hz, 1H); 3.75-3.84 (m, 1H); 3.54 (s, 3H); 3.31-3.42 (m, 1H); 3.21-3.31 (m, 1H); 2.62 (s, 3H); 2.47-2.56 (m, 1H); 1.46-1.92 (m, 7H); 1.06-1.14 (m, 1H).

### Example 8

### (3aR,4S,7aR)-methyl 4-amino-4-(m-tolylethynyl)octahydro-1H-indole-1-carboxylate

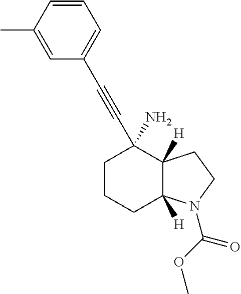 [SMILES: COC(=O)N1CC[C@@H]2[C@H]1CCC[C@@]2(N)C#Cc1cccc(C)c1]

A solution of (3aR,7aR)-methyl 4-(4-methoxybenzylamino)-4-(m-tolylethynyl)octahydro-1H-indole-1-carboxylate (intermediate C, 800 mg, 1.85 mmol) in CH₃CN/H₂O (40 mL/10 mL) was treated with an aq. soln. of (NH₄)₂Ce(NO₃)₆ (2.02 g, 3.70 mmol) in CH₃CN/H₂O (40 mL/10 mL). The mixture was stirred for 2 h at RT, poured onto a sat. aq. soln. of NaHCO₃ (100 mL) and extracted with AcOEt (2*1000 mL). The combined org. phases were dried over Na₂SO₄, filtered and concentrated in vacuo. Purification by flash chromatography (SiO₂, Heptane/AcOEt/MeOH 100:0:0 to 0:90:10) and recrystallization in heptane/AcOEt (98:2) provided (3aR,4S,7aR)-methyl 4-amino-4-(m-tolylethynyl)octahydro-1H-indole-1-carboxylate (90 mg, 16% yield). UPLC/MS: rt=0.81 min; found 313.0 [M+H⁺]. ¹H-NMR (400 MHz, d₆-DMSO): 7.13-7.24 (m, 4H); 3.73-3.85 (m, 1H); 3.53 (s, 3H); 3.33-3.40 (m, 1H); 3.19-3.29 (m, 1H); 2.39-2.48 (m, 1H); 2.27 (s, 3H); 2.06 (br s, 2H); 1.79-1.87 (m, 3H); 1.43-1.61 (m, 4H); 1.01-1.08 (m, 1H).

### Example 9

### (3aR,4S,7aR)-ethyl 4-amino-4-(m-tolylethynyl)octahydro-1H-indole-1-carboxylate

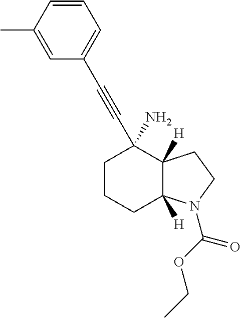 [SMILES: CCOC(=O)N1CC[C@@H]2[C@H]1CCC[C@@]2(N)C#Cc1cccc(C)c1]

Example 9 can be prepared by analogy to Example 9, starting from (3aR,7aR)-ethyl 4-oxooctahydro-1H-indole-1-carboxylate. UPLC/MS: rt=0.79 min; found 327.1 [M+H⁺]. ¹H-NMR (400 MHz, d₆-DMSO): 7.13-7.24 (m, 4H); 3.93-4.05 (m, 2H); 3.75-3.85 (m, 1H); 3.33-3.38 (m, 1H); 3.17-3.27 (m, 1H); 2.27 (s, 3H); 2.08 (br s, 2H); 1.76-1.87 (m, 3H); 1.43-1.62 (m, 4H); 1.12-1.16 (m, 3H); 1.03-1.10 (m, 1H).

### Physico-Chemical Properties

Solubility of compounds of the invention was determined in the following buffer: Cl-free phosphate buffer (0.067 M) is made from KH₂PO₄ solution and titrated to pH 6.8 with NaOH. The table below represents solubility values of compounds of the invention in the above buffer.

| Example | Solubility (g/l) |
|---|---|
| 1 | 0.103 |
| 2 | >0.3 |
| 3 | >0.3 |
| 4 | n.t. |
| 5 | 0.175 |
| 6 | 0.153 |
| 7 | >0.3 |
| 8 | >0.3 |
| 9 | >0.3 |

### Biological Testing

Activity of compounds of the present invention was examined by determination to what extent the glutamate-induced elevation of the intracellular calcium concentration in L(tk-) cells expressing human mGluR5a receptors (see L. P. Daggett et al., Neuropharm. Vol. 34, pages 871-886, 1995) is inhibited by utilizing methods as described e.g. by L. P. Daggett et al., Neuropharm. Vol. 34, pages 871-886 (1995) and P. J. Flor et al., J. Neurochem. Vol. 67, pages 58-63 (1996).

The table below represents IC₅₀ values of the inhibition of the glutamate-induced elevation of the intracellular calcium concentration.

| Example | IC₅₀ (nM) |
|---|---|
| 1 | 16 |
| 2 | 1100 |
| 3 | 530 |
| 4 | 1430 |
| 5 | 146 |
| 6 | 740 |
| 7 | 470 |
| 8 | 330 |
| 9 | 710 |

The following are further embodiments of the invention:

### Embodiment 1

A compound of the formula I

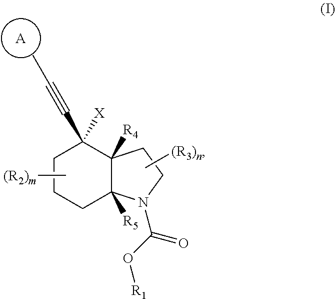

or a salt thereof, wherein
R₁ is C₁₋₄alkyl, C₃₋₆cycloalkyl or C₃₋₆cycloalkyl-C₁₋₄alkyl;
R₂ and R₃ independently are halogen, cyano, hydroxy, amino, C₁₋₄alkyl; C₁₋₄halogenalkyl; C₁₋₄hydroxyalkyl; C₁₋₄-aminoalkyl; C₁₋₄alkylamino-C₁₋₄alkyl; di-(C₁₋₄alkyl)amino-C₁₋₄alkyl; C₁₋₄alkoxy-C₁₋₄alkyl; C₂₋₄alkenyl; C₂₋₄halogenalkenyl; C₂₋₄alkinyl; C₂₋₄halogenalkinyl; C₁₋₄alkoxy; C₁₋₄halogenalkoxy; C₁₋₄alkyl-amino; di-(C₁₋₄alkyl)amino or C₃₋₆cycloalkyl, wherein one carbon atom of the C₃₋₆cycloalkyl may be replaced by an oxygen atom and wherein the C₃₋₆cycloalkyl may be attached directly to the ring system or via a C₇₋₂alkylene or an oxygen;
m is 0, 1, 2, 3, 4, 5 or 6;
n is 0, 1, 2, 3 or 4;
R₄ and R₅ independently are hydrogen, halogen or methyl;
X is hydroxy or amino;
A is selected from

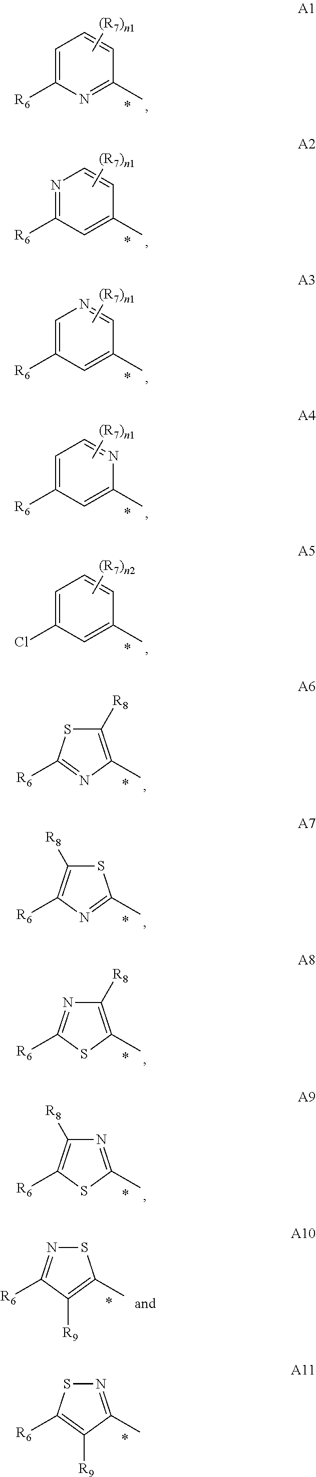

wherein the bond marked with the asterisk is attached to the ethynyl-moiety;
R₆ is hydrogen, halogen, methyl or halogenmethyl;
R₇ is halogen, hydroxy, amino, cyano, methyl or methoxy;
R₈ is hydrogen, halogen, hydroxy, amino, cyano, methyl or methoxy;
n1 is 0, 1, 2 or 3; and
n2 is 0, 1, 2, 3 or 4.

### Embodiment 2

A compound of formula I according to claim 1, wherein R₁ is C₁₋₄alkyl; or a salt thereof.

### Embodiment 3

A compound of formula I according to any of claim 1 or 2, wherein m and n are both 0; and R₄ and R₅ are both hydrogen; or a salt thereof.

### Embodiment 4

A compound of formula I according to any of claims 1-3, wherein X is hydroxy; or a salt thereof.

### Embodiment 5

A compound of formula I according to any of claims 1-4, wherein A is A2; or a salt thereof.

### Embodiment 6

A compound of formula I according to any of claims 1-4, wherein A is A5; or a salt thereof.

### Embodiment 7

A compound of formula I according to claim 1, wherein R₁ is C₁₋₄alkyl; m and n are both 0;

R₄ and R₅ are both hydrogen; X is hydroxy; A is A2; wherein the bond marked with the asterisk is attached to the ethynyl-moiety; R₆ is chloro, methyl, fluoromethyl, difluoromethyl or trifluoromethyl; R₇ is fluoro; and n1 is 0, 1 or 2; or a salt thereof.

### Embodiment 8

Further examples of suitable compounds of the invention are compounds selected from the following group P:

### Group P: Suitable Compounds of the Invention:

(3aR,4S,7aR)-methyl 4-((3-chlorophenyl)ethynyl)-4-hydroxyoctahydro-1H-indole-1-carboxylate;
(3aR,4S,7aR)-methyl 4-hydroxy-4-((5-methylpyridin-3-yl)ethynyl)octahydro-1H-indole-1-carboxylate;
(3aR,4S,7aR)-methyl 4-hydroxy-4-((2-methylpyridin-4-yl)ethynyl)octahydro-1H-indole-1-carboxylate;
(3aR,4S,7aR)-methyl 4-hydroxy-4-((6-methylpyridin-2-yl)ethynyl)octahydro-1H-indole-1-carboxylate;
(3aR,4S,7aR)-methyl 4-((2-chloropyridin-4-yl)ethynyl)-4-hydroxyoctahydro-1H-indole-1-carboxylate;
(3aR,4S,7aR)-methyl 4-hydroxy-4-((4-methylpyridin-2-yl)ethynyl)octahydro-1H-indole-1-carboxylate;
(3aR,4S,7aR)-methyl 4-hydroxy-4-((2-methylthiazol-4-yl)ethynyl)octahydro-1H-indole-1-carboxylate;
(3aR,4S,7aR)-methyl 4-amino-4-(m-tolylethynyl)octahydro-1H-indole-1-carboxylate; or
(3aR,4S,7aR)-ethyl 4-amino-4-(m-tolylethynyl)octahydro-1H-indole-1-carboxylate; or salts of these compounds.

### Embodiment 9

A pharmaceutical composition comprising a therapeutically effective amount of a compound according to any one of embodiments 1 to 8 and one or more pharmaceutically acceptable carriers.

### Embodiment 10

A combination comprising a therapeutically effective amount of the compound according to any one of embodiments 1 to 8 and one or more therapeutically active agents.

### Embodiment 11

A method of inhibiting mGluR5 activity in a subject, wherein the method comprises administering to the subject a therapeutically effective amount of the compound according to any one of embodiments 1 to 8.

### Embodiment 12

A method of treating a disorder or a disease in a subject mediated by mGluR5, wherein the method comprises administering to the subject a therapeutically effective amount of a compound according to any one of embodiments 1 to 8. Preferably said disorder or said disease is selected from L-dopa induced dyskinesias in Parkinsons Disease and fragile X syndrome.

### Embodiment 13

A compound according to any one of embodiments 1 to 8, for use as a medicament.

### Embodiment 14

Use of a compound according to any one of embodiments 1 to 8, for the treatment of a disorder or disease in a subject mediated by mGluR5.

### Embodiment 15

Use of a compound according to any one of embodiments 1 to 8, for the treatment of a disorder or disease in a subject characterized by an abnormal activity of mGluR5. Preferably said disorder or said disease is selected from L-dopa induced dyskinesias in Parkinsons Disease and fragile X syndrome.

1. A compound of the formula I 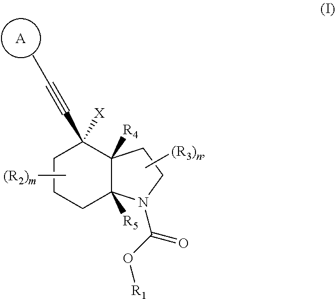
or a salt thereof, wherein
R₁ is C₁₋₄alkyl, C₃₋₆cycloalkyl or C₃₋₆cycloalkyl-C₁₋₄alkyl;
R₂ and R₃ independently are halogen, cyano, hydroxy, amino, C₁₋₄alkyl; C₁₋₄halogenalkyl; C₁₋₄hydroxyalkyl; C₁₋₄aminoalkyl; C₁₋₄alkylamino-C₁₋₄alkyl; di-(C₁₋₄alkyl)amino-C₁₋₄alkyl; C₁₋₄alkoxy-C₁₋₄alkyl; C₂₋₄alkenyl; C₂₋₄halogenalkenyl; C₂₋₄alkinyl; C₂₋₄halogenalkinyl; C₁₋₄alkoxy; C₁₋₄halogenalkoxy; C₁₋₄alkyl-amino; di-(C₁₋₄alkyl)amino or C₃₋₆cycloalkyl, wherein one carbon atom of the C₃₋₆cycloalkyl may be replaced by an oxygen atom and wherein the C₃₋₆cycloalkyl may be attached directly to the ring system or via a C₁₋₂alkylene or an oxygen;
m is 0, 1, 2, 3, 4, 5 or 6;
n is 0, 1, 2, 3 or 4;
R₄ and R₅ independently are hydrogen, halogen or methyl;
X is hydroxy or amino;
A is selected from 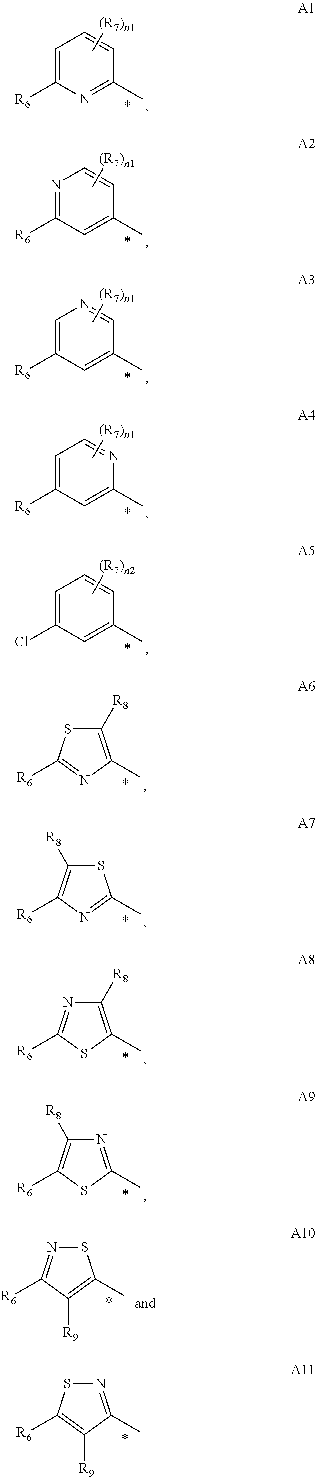
wherein the bond marked with the asterisk is attached to the ethynyl-moiety;
R₆ is hydrogen, halogen, methyl or halogenmethyl;
R₇ is halogen, hydroxy, amino, cyano, methyl or methoxy;
R₈ is hydrogen, halogen, hydroxy, amino, cyano, methyl or methoxy;
n1 is 0, 1, 2 or 3; and
n2 is 0, 1, 2, 3 or 4.

2. A compound of formula I according to claim 1, wherein R₁ is C₁₋₄alkyl; or a salt thereof.

3. A compound of formula I according to claim 1, wherein m and n are both 0; and R₄ and R₅ are both hydrogen; or a salt thereof.

4. A compound of formula I according to claim 1, wherein X is hydroxy; or a salt thereof.

5. A compound of formula I according to claim 1, wherein A is A2; or a salt thereof.

6. A compound of formula I according to claim 1, wherein A is A5; or a salt thereof.

7. A compound of formula I according to claim 1, wherein R₁ is C₁₋₄alkyl; m and n are both 0;
R₄ and R₅ are both hydrogen; X is hydroxy; A is A2; wherein the bond marked with the asterisk is attached to the ethynyl-moiety; R₆ is chloro, methyl, fluoromethyl, difluoromethyl or trifluoromethyl; R₇ is fluoro; and n1 is 0, 1 or 2; or a salt thereof.

8. A compound of formula I according to claim 1, wherein said compound is selected from the group consisting of
(3aR,4S,7aR)-methyl 4-((3-chlorophenyl)ethynyl)-4-hydroxyoctahydro-1H-indole-1-carboxylate;
(3aR,4S,7aR)-methyl 4-hydroxy-4-((5-methylpyridin-3-yl)ethynyl)octahydro-1H-indole-1-carboxylate;
(3aR,4S,7aR)-methyl 4-hydroxy-4-((2-methylpyridin-4-yl)ethynyl)octahydro-1H-indole-1-carboxylate;
(3aR,4S,7aR)-methyl 4-hydroxy-4-((6-methylpyridin-2-yl)ethynyl)octahydro-1H-indole-1-carboxylate;
(3aR,4S,7aR)-methyl 4-((2-chloropyridin-4-yl)ethynyl)-4-hydroxyoctahydro-1H-indole-1-carboxylate;
(3aR,4S,7aR)-methyl 4-hydroxy-4-((4-methylpyridin-2-yl)ethynyl)octahydro-1H-indole-1-carboxylate;
(3aR,4S,7aR)-methyl 4-hydroxy-4-((2-methylthiazol-4-yl)ethynyl)octahydro-1H-indole-1-carboxylate;
(3aR,4S,7aR)-methyl 4-amino-4-(m-tolylethynyl)octahydro-1H-indole-1-carboxylate; and
(3aR,4S,7aR)-ethyl 4-amino-4-(m-tolylethynyl)octahydro-1H-indole-1-carboxylate;
or salts of these compounds.

9. A pharmaceutical composition comprising a therapeutically effective amount of a compound according to claim 1 and one or more pharmaceutically acceptable carriers.

10. A combination comprising a therapeutically effective amount of the compound according to claim 1 and one or more therapeutically active agents.

11. A method of inhibiting mGluR5 activity in a subject, wherein the method comprises administering to the subject a therapeutically effective amount of the compound according to claim 1.

12. A method of treating a disorder or a disease in a subject mediated by mGluR5, wherein the method comprises administering to the subject a therapeutically effective amount of a compound according to claim 1.

13-15. (canceled)
